(19) United States
(12) Patent Application Publication
(10) Pub. No.: US 2003/0198581 A1
(43) Pub. Date: Oct. 23, 2003

(54) Sterilization container

(76) Inventors: Ralph B. Sweet II, Warsaw, IN (US); Shane E. Maust, Milford, IN (US)

Correspondence Address: BAKER & DANIELS, 111 E. WAYNE STREET, SUITE 800, FORT WAYNE, IN, 46802

(21) Appl. No.: 10/126,085

(22) Filed: Apr. 19, 2002

(51) Int. Cl. A61L 2/16

(52) U.S. Cl. 422/292; 220/812; 220/826

(57) ABSTRACT

A sterilization container having a lid which remains secured to the container in its open and closed positions and throughout a range of motion between the open and closed positions. The container includes a bottom wall and sidewalls to define an interior space. The lid is positioned near an upper limit of the interior space when closed and positioned parallel and proximate a sidewall when open. The lid is mounted to the container for both pivotal and sliding movement. The container may have an upper panel for supporting bone screws and a stop for engaging the lid. A tab extending from the lid may be engaged by a removable cover mounted to the container over the lid to bias the lid into engagement with the stop. The container includes a plurality of openings to allow the entry of a sterilant, such as steam, into the interior of the container.

### BACKGROUND OF THE INVENTION

1. Field of the Invention

The present invention relates to sterilization containers, more specifically, to sterilization containers which may used to hold surgical items such as bone screws.

2. Description of the Related Art

A variety of sterilization containers for holding surgical items are known in the art. Oftentimes, such containers include lids to retain the surgical items in the container during sterilization procedures. It is also known to include indicia on such lids which provide information about the sterilized items held in the container so that such information is available when the items are later removed for use.

When opening sterilization containers having lids, the lids may be completely removable from the container. One drawback to such removable lids is that completely removable lids may be misplaced. Sterilization container lids may also be configured to have open positions wherein the lids project laterally outwardly from the sidewalls of the container in a shelf-like configuration. One drawback to such outwardly projecting lids is that such lids take up additional space which is undesirable when such containers are used to hold items in an operating room environment.

### SUMMARY OF THE INVENTION

The present invention provides a sterilization container having an improved lid. The lid remains secured to the container in both its open and closed position and throughout its range of motion between these two positions. In the open position, the lid is positioned parallel to and near one of the sidewalls of the container to minimize the space required by the container when it is used to hold sterilized items so that such items are readily accessible for use.

The invention comprises, in one form thereof, a sterilization container having a bottom wall and a plurality of sidewalls which may be positioned transverse to the bottom wall. The bottom and sidewalls define an interior space in which items may be placed during sterilization procedures. A substantially planar lid is mounted to the container and is moveable through a range of motion between a closed position and an open position. In the closed position, the lid is at least a portion of the interior space of the container. The lid may be positioned transverse to the sidewalls when it is in the closed position. When in the open position, the lid is positioned substantially parallel and proximate to one of the sidewalls of the container. The lid is secured to the container when it is in the open and closed positions and throughout the range of motion between these two positions. The container includes a plurality of openings to provide access to the interior space for a sterilant, for example, steam.

The sterilization container may have a lid which is both pivotally and slidably mounted to the container. The container may have two vertical slots and the lid have two projections disposed within the slots to thereby mount the lid in a pivotal and sliding manner.

The sterilization container may also include an upper panel having apertures for holding items, such as bone screws, within the interior space of the container. A stop may be mounted on the upper panel to engage the lid when the lid is in a closed position. The lid may further include an outwardly extending tab projecting substantially transverse to a major surface of said lid and engageable by a cover removably securable to the container. Engagement of the cover and tab biases the lid into engagement with the stop on the upper panel and thereby firmly holds the lid in place when the cover is mounted to the container.

An advantage of the present invention is that it provides a sterilization container having a lid which remains secured to the container both in its open and closed positions and throughout the range of motion of the lid between the open and closed positions and thereby prevents the misplacement of the lid during such opening and closing movements.

Another advantage is that the positioning of the lid parallel and proximate to one of the sidewalls of the container when the lid is in an open position minimizes the space occupied by the container when the lid of the container is placed in an open position.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is an exploded perspective view of the present invention.

FIG. 2 is another exploded perspective view of a container in accordance with the present invention.

FIG. 3 is a perspective view of a container.

FIG. 4 is a perspective view of a container.

FIG. 5 is a perspective view of a container.

FIG. 6 is a perspective view of a container.

FIG. 7 is a partial perspective view of a container showing a lid in an open position.

FIG. 8 is a partial perspective view of a container showing a lid in a partially open position.

FIG. 9 is a perspective view of stop member.

Corresponding reference characters indicate corresponding parts throughout the several views. Although the exemplification set out herein illustrates an embodiment of the invention, in one form, the embodiment disclosed below is not intended to be exhaustive or to be construed as limiting the scope of the invention to the precise form disclosed.

### DESCRIPTION OF THE PRESENT INVENTION

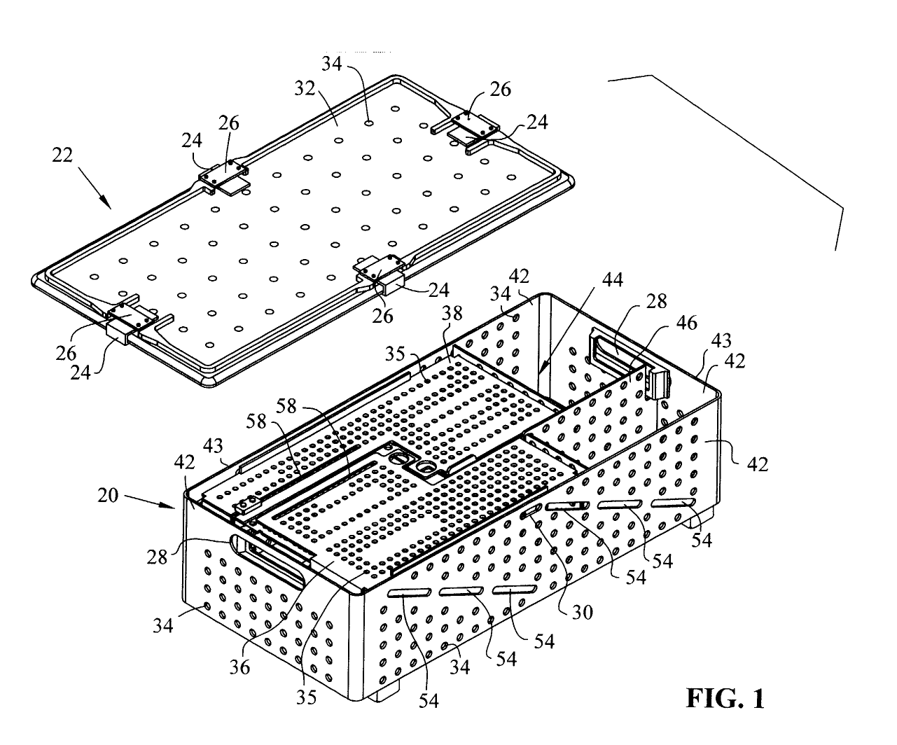

Referring now to the drawings and particularly to FIG. 1, there is shown a sterilization container 20 in accordance with the present invention and a cover 22 which can be removably mounted to container 20.

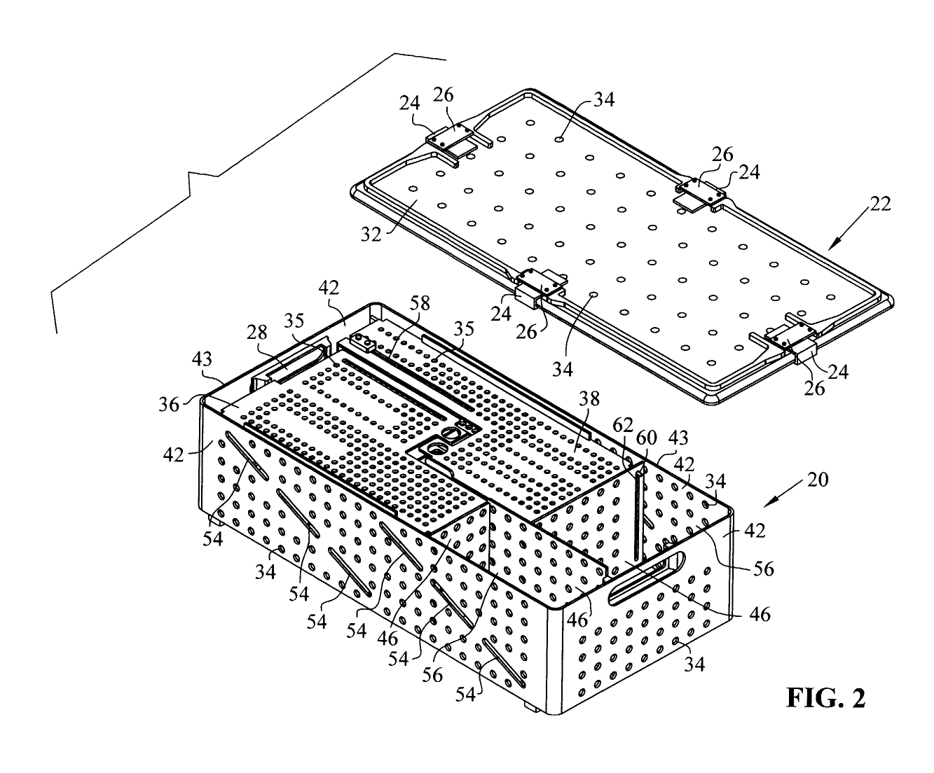

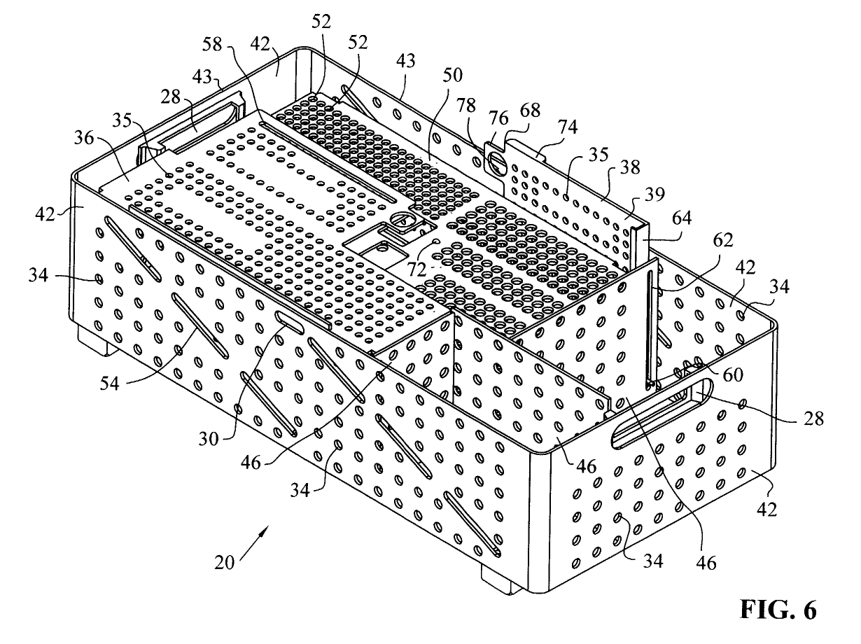

Cover 22 includes clamps 24 which are secured to cover 22 by retainers 26 which are riveted to main body 32 of cover 22. Clamps 24 can slide inwardly and outwardly to grasp and release container 20. In FIGS. 1 and 2, clamps 24 are shown at their innermost position. To removably mount cover 22 to container 20, clamps 24 can be slid outwardly, cover 22 positioned on the top of container 20, and clamps 24 can then be slid inwardly. Clamps 24 engage container 20 at handle openings 28 and clamp openings 30. Clamp openings 30 are positioned on two sides of container 20 and are shown in FIGS. 1 and 6. Clamp openings 30 are not illustrated in FIGS. 2-5. Main body 32 of cover 22 includes openings 34 which permit a sterilant, such as steam, to enter the container when cover 22 is mounted to the container 20. The main body 32 of cover can be manufactured of a transparent plastic material to allow visual access through cover 22 when it is mounted to container 20. Illustrated container 20 is primarily formed out of stainless steel and aluminum but alternative materials can also be employed.

The illustrated container 20 includes two lids 36, 38, however, alternative embodiments may have only one lid or may have more than two lids. The illustrated container 20 also includes a bottom wall 40 and sidewalls 42. The bottom wall 40 is substantially planar and sidewalls 42 extend transverse to the bottom wall 40 at each of the edges of the bottom wall 40 to form an enclosure having an opening defined by the upper edges 43 of the sidewalls 42. Together, the bottom wall 40 and sidewalls 42 thereby define an interior space 44 of container 20. Openings 34 are provided in bottom wall 40 and sidewalls 42 to facilitate the entry of a sterilant to interior space 44 of container 20.

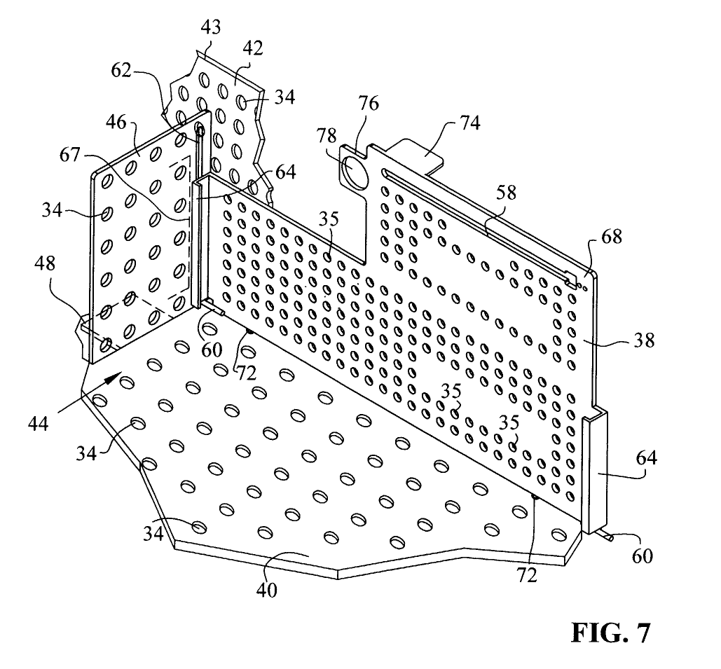

The illustrated container 20 also includes partitions 46 which subdivide interior space 44. Partitions 46 may be riveted together and have bent tab feet 48, seen in FIG. 7, which are riveted to bottom wall 40 to secure partitions 46 to container 20. Alternative methods of securing partitions 46 together and to container 20, such as welding, could alternatively be used. Also secured to partitions 46 is upper panel 50. Upper panel 50 is located in interior space 44 near its upper boundary and includes a plurality of apertures 52. Apertures 52 are sized to suspend bone screws (not shown) within interior space 44. Elongate, oval openings 54 may be located in side panels 42 to facilitate visual access to the bone screws suspended within interior space 44. Openings 54 may be advantageously positioned to generally track the position of the tips of the suspended bone screws. By positioning upper panel 50 over only a portion of the open area defined by the upper edges 43 of sidewalls 42, compartments 56 may be used to hold instruments or other items. Shelf-like brackets (not shown) can also be mounted in compartments 56 to facilitate the positioning of small instrument trays within compartments 56.

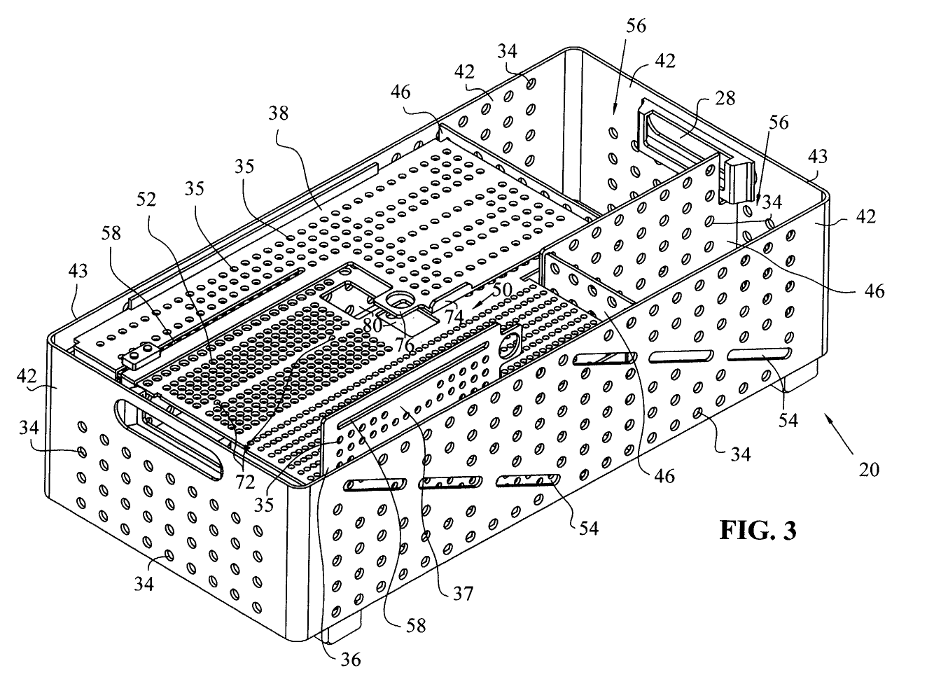

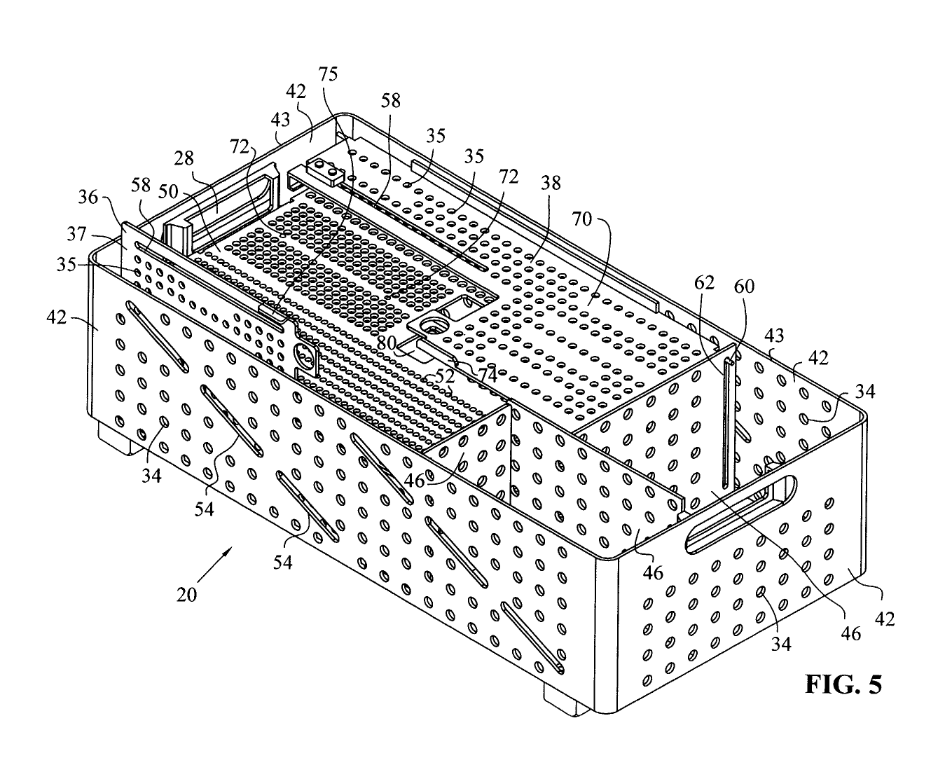

Lids 36, 38 are pivotally and slidably mounted to container 20. In FIGS. 1 and 2, lids 36 and 38 are both in closed positions wherein lids 36 and 38 are positioned opposite the bottom wall 40 proximate an upper limit of a portion of interior space 44. In FIGS. 3 and 5, lid 36 is in an open position wherein lid 36 is positioned substantially parallel to and proximate a sidewall 42. In the illustrated embodiment, lid 36 in its open position, is partially disposed within interior space 44. L-shaped lid 36 also includes a portion 37 which extends outwardly of container 20, i.e., portion 37 that extends upwardly of edges 43, when lid 36 is placed in its open position. As can be seen in FIGS. 3 and 5, although portion 37 extends outwardly of container 20 it does not project horizontally outwardly of sidewalls 42 and thereby does not enlarge the footprint occupied by container 20 when lid 36 is placed in its open position to provide access to the screws or other items held within container 20 proximate lid 36.

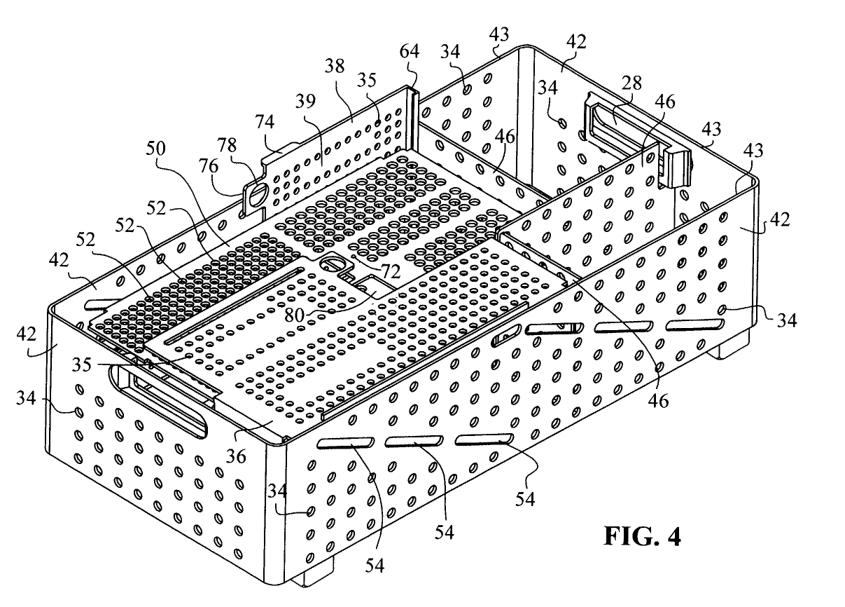

Similarly, lid 38 is substantially parallel and proximate a sidewall 42 when it is placed in an open position as illustrated in FIGS. 4 and 6. Lid 38 is also partially disposed within interior space 44 when it is in an open position and has a portion 39 extending upwardly from container 20. Lid portions 37, 39 which remain visible when lids 36, 38 are, respectively, placed in an open position can have informational indicia located thereon concerning the items which are made accessible when the lid is placed in an open position. For example, container could be used to house both large and small fragment screws wherein the large fragment selection is placed under one of the lids and the small fragment selection is placed under the other lid. Lid portions 37, 39 could be color coded so that it would be clear which selection of screws was being presented when a lid was opened. The informational indicia on portions 37, 38 could also take the form of textual information. Another form of informational indicia commonly found on screw cases is a screw gauge, and lids 36, 38 each may include a screw gauge 58 as schematically illustrated in FIG. 1.

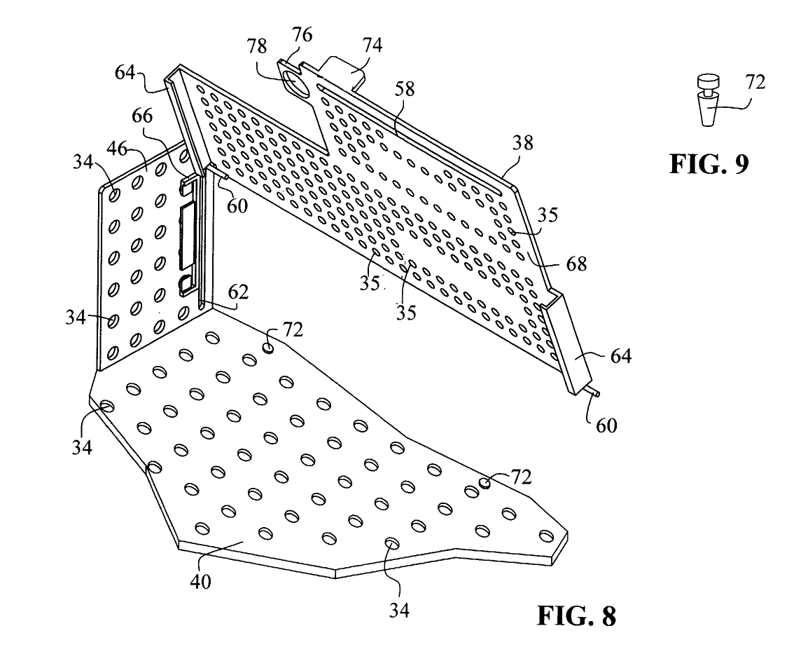

As most easily seen in FIGS. 7 and 8, lid 38 includes two projections 60 disposed at opposite sides of lid 38. The illustrated projections 60 are metal rods which are welded to lid 38. Projections 60 are disposed in vertical slots 62 to slidingly and pivotally mount lid 38 to container 20. In the illustrated embodiment, slots 62 are located in partitions 46, as can also be seen in FIGS. 5 and 6, however, slots 62 could alternatively be located on container 20. The location of slots and rods could also be reversed to slidingly and pivotally mount the lids to the container whereby the slots would be located on lids 36, 38 and the projections would be mounted on container 20.

As shown in FIGS. 7 and 8, lid 38 includes C-shaped rails 64 at two edges. Rails 64 provide added rigidity to lid 38. Lids 36, 38 each also include a plurality of openings 35. Openings 35 may be positioned on lids 36, 38 to be positioned above apertures 52 in upper panel 50 when lids 36, 38 are closed. Openings 35 allow for the passage of a sterilant, such as steam, therethrough.

C-shaped brackets 66 are located adjacent slots 62 to control the position of lids 36, 38 as lids 36, 38 are moved through a range of motion between their open and closed positions. As can be seen with reference to FIG. 8, lid 38 can be rotated when projections 60 are located near the top of slots 62 as when the lid is in the closed position. Once lid 38 is rotated to a position where its major planar surfaces 68 and 70 are vertically oriented, lid 38 can be slid downwardly whereby a portion of lid 38 will be disposed within interior space 44 and lid 38 will be positioned substantially parallel and proximate a sidewall 42 as shown in FIGS. 6 and 7. As projections 60 are slid downwardly within slot 62 it will be positioned between sidewall 42 and C-bracket 66 and may no longer rotate freely. C-bracket 66 will engage rail 64 to prevent rotation in one direction and the sidewall 42 located proximate lid 38 will prevent rotation in the opposite direction. Although C-bracket 66 is not shown in FIG. 7, a dashed outline 67 in FIG. 7 illustrates where C-bracket 66 is located to prevent rotation of lid 38. The upper corner of C-bracket 66 may be rounded to thereby provide a shoulder against which rail 64 may bear as it is rotated between a vertical and a horizontal orientation.

One or more stops 72 are mounted on upper panel 50 for engagement with lids 36, 38. Stops 72, shown in FIG. 9, have a pointed section which may be inserted through an opening in upper panel 50 to thereby mount the stop 72. Stops 72 may be made of silicone and are known in the art. When lid 38 is placed in its closed position, major surface 68 engages a stop 72 and positions lid 38 at a distance above upper panel 50. Similarly two stops 72 are used to engage lid 36 when it is placed in its closed position. Stops 72 space lids 36, 38 a distance above upper panel 50 that is greater than the thickness of the screw heads located above upper panel 50 when screws are suspended from panel 50. This prevents lids 36, 38 from directly engaging the screws held within interior space 44 and thereby reduce the possibility of introducing unwanted substances, such as condensation, onto the screws. Lids 36, 38, however, are positioned close enough to the heads of the screws supported by panel 50 to prevent the screws from withdrawing from apertures 52 when lids 36, 38 are in the closed position.

Additional stops 72 are located on the bottom panel 40 to support the bottom edge of lids 36, 38 when lids 36, 38 are placed in their open positions. By using stops 72 on bottom panel 40, the stops 72 can reduce the noise created when lids 36, 38 are placed in their open positions and can also be used to support the weight of lids 36, 38 so that projections 60 are not used to support the weight of lids 36, 38 when they are in the open position.

The use of projections 60 and slots 62 has the potential to lead to racking or binding of lid 38 if it is slid in a manner which positions projections 60 at different vertical heights. To minimize the chances of the occurrence of such binding and simultaneously avoid the introduction of an expensive gearing mechanism, lid 38 uses gripping element 74 which takes the form of a bent tab in the illustrated embodiment. Gripping element 74 is positioned above the center of mass of lid 38 when lid 38 is located in a vertically oriented position as shown in FIG. 7. Thus, when a user of container 20 seeks to slide lid 38, the position of gripping element 74 will encourage the user to grip lid 38 above its center of mass. By gripping lid 38 directly above its center of mass, the lid 38 is less likely pivot to bring the center of mass into vertical alignment with the location at which the lid is supported. Element 74 performs another function when lid 38 is in the closed position wherein major planar surface 60 is facing the interior space 44 and major planar surface 70 is facing upwardly. The upper edge of element 75 is engaged by the bottom surface of main body 32 of cover 22 when lid 38 is placed in the closed position and cover 22 is mounted to container 20. The engagement of element 75 by cover 22 biases lid 38 into engagement with stop 72 to thereby firmly secure lid 38 in place and thereby minimize unwanted movement of lid 38 during transportation and handling of container 20 and keep lid 38 in a position where it maintains screws held in container 20 in their respective apertures 52.

Lid 36 is shown, in FIG. 5, having a gripping element 75 which takes the form of a bent tab which functions in the same manner as element 74 with regard to the sliding of lid 36 and the securement of lid 36 in its closed position. The remaining Figures do not illustrate element 75 on lid 36.

Lids 36 and 38 also include finger grips 76 which define a center opening 78 through which a finger can be inserted to grip lids 36, 38 when they are in their closed positions to pivot lids 36, 38 upwardly prior to sliding lids 36, 38 vertically downward to reach their open positions. Upper panel 50 includes two openings 80 positioned proximate finger grips 76 when lids 36, 38 are in their closed positions to thereby provide clearance for a finger inserted through opening 78. See FIG. 3.

While this invention has been described as having an exemplary design, the present invention may be further modified within the spirit and scope of this disclosure. This application is therefore intended to cover any variations, uses, or adaptations of the invention using its general principles.

What is claimed is:

1. A sterilization container comprising:
a bottom wall;
a plurality of sidewalls extending from said bottom wall, said bottom wall and plurality of sidewalls defining an interior space;
a substantially planar lid mounted to said container, said lid being moveable through a range of motion between a closed position and an open position, wherein said lid in said closed position is positioned opposite said bottom wall and proximate an upper limit of at least a portion of said interior space and said lid in said open position is positioned substantially parallel and proximate a first one of said side walls, said lid being secured to said container in said closed and open positions and throughout said range of motion; and
wherein said container defines a plurality of openings providing sterilant access to said interior space when said lid is in said closed position.

2. The sterilization container of claim 1 wherein at least a portion of said lid is disposed within said interior space in said open position.

3. The sterilization container of claim 1 wherein said container further comprises a second lid mounted to said container, said second lid being moveable through a range of motion between a closed position and an open position, wherein said second lid in said closed position is positioned opposite said bottom wall and defines an upper limit of at least a second portion of said interior space and said second lid in said open position is positioned substantially parallel and proximate to a second one of said side walls, said second lid being secured to said container in said closed and open positions and throughout said range of motion.

4. The sterilization container of claim 1 wherein said lid is both pivotally and slidingly mounted to said container.

5. The sterilization container of claim 4 wherein said lid has a center of gravity and includes a gripping element positioned vertically above said center of gravity when said lid is in said open position.

6. The sterilization container of claim 1 wherein said container defines first and second vertically oriented slots and said lid further comprises first and second projections pivotally and slidably disposed within said first and second slots respectively.

7. The sterilization container of claim 6 further comprising first and second brackets respectively mounted to said first and second partition walls and engageable with said lid wherein said brackets prevent pivotal movement of said lid through a portion of said range of motion.

8. The sterilization container of claim 1 wherein said container further comprises an upper panel mounted within said interior space and defining a plurality of apertures adapted to suspend items within said interior space.

9. The sterilization container of claim 1 further comprising a cover removably securable to said container in a position which inhibits access to said lid and said interior space.

10. The sterilization container of claim 9 wherein said lid defines a first major surface which faces said interior space when said lid is in said closed position and an oppositely disposed second major surface and said container further comprises an upper panel mounted within said interior space, said upper panel defining a plurality of apertures adapted to suspend items within said interior space and having at least one stop engageable with said lid when said lid is in said closed position, said lid further comprising a tab extending substantially transverse and outwardly from said second major surface and wherein mounting of said removable cover to said container with said lid in said closed position engages said cover with said tab and biases said lid into engagement with said stop.

11. The sterilization container of claim 10 wherein said lid defines a center of gravity and said tab defines a gripping element, said tab being positioned vertically above said center of gravity when said lid is in said open position.

12. A sterilization container comprising:
a bottom wall;
a plurality of substantially planar sidewalls extending substantially transverse to said bottom wall, said bottom wall and said plurality of side walls defining an interior space;
a substantially planar lid slidably and pivotally mounted to said container, said lid being moveable through a range of motion between a closed position and an open position wherein said lid in said closed position is positioned opposite said bottom wall, substantially transverse to said sidewalls and proximate an upper limit of at least a portion of said interior space, said lid in said open position is positioned substantially parallel and proximate a first one of said side walls, said lid being secured to said container in said closed and open positions and throughout said range of motion; and
wherein said container defines a plurality of openings providing sterilant access to said interior space when said lid is in said closed position.

13. The sterilization container of claim 12 wherein at least a portion of said lid is disposed within said interior space when said lid is in said open position and said lid is both pivotally and slidingly mounted to said container.

14. The sterilization container of claim 12 further comprising:
an upper panel mounted within said interior space, said upper panel defining a plurality of apertures adapted to suspend items within said interior space, said upper panel positioned substantially parallel to said bottom wall;
a second lid mounted to said container, said second lid being moveable through a range of motion between a closed position and an open position, wherein said second lid in said closed position is positioned opposite said bottom wall and defines an upper limit of at least a second portion of said interior space and said second lid in said open position is positioned substantially parallel and proximate to a second one of said side walls, said second lid being secured to said container in said closed and open positions and throughout said range of motion; and
wherein each of said first and second lids are positioned to overlay at least a portion of said upper panel when said first and second lids are each in said closed positions.

15. The sterilization container of claim 14 further comprising:
a cover removably securable to said container in a position which inhibits access to said lid and said interior space;
first and second stops disposed on said upper panel, said first and second stops respectively engageable with said first and second lids when said first and second lids are in said closed positions; and
first and second tabs respectively disposed on said first and second lids, each of said first and second tabs extending substantially perpendicular with respect to said upper panel when said first and second lids are in said closed positions, mounting of said cover to said container engaging said cover with said first and second tabs and biasing said first and second lids into respective engagement with said first and second stops.

16. The sterilization container of claim 14 wherein said first lid further comprises a finger grip and said upper panel includes an opening and said opening is positioned proximate said finger grip when said first lid is closed whereby said opening provides clearance for use of said finger grip.

17. The sterilization container of claim 14 wherein said container defines first and second vertically oriented slots and further comprises at least one partition member disposed within said interior space and defining one of said vertically oriented slots, said first lid further comprising first and second projections respectively disposed within said first and second vertically oriented slots.

18. The sterilization container of claim 17 wherein said first lid has a center of gravity and further comprises a gripping element disposed vertically above said center of gravity when said first lid is in said open position.

19. The sterilization container of claim 17 wherein at least a portion of each of said first and second lids are disposed within said interior space when said first and second lids are in said open positions.
